(19) United States
(12) Patent Application Publication
(10) Pub. No.: US 2024/0057957 A1
(43) Pub. Date: Feb. 22, 2024

(54) ENERGY DISPERSIVE X-RAY SPECTROSCOPY SENSING UNIT

(71) Applicant: Applied Materials Israel Ltd., Rehovot (IL)

(72) Inventors: Martin Chauvin, Rehovot (IL); Eugene Brozgol, Rehovot (IL); Marat Feldman, Rehovot (IL); Yosef Basson, Mazkeret-Batya (IL); Itay Asulin, Rehovot (IL); Shmuel Nakash, Rehovot (IL); Jacob Levin, Rehovot (IL)

(73) Assignee: Applied Materials Israel Ltd., Rehovot (IL)

(21) Appl. No.: 18/110,328

(22) Filed: Feb. 15, 2023

Related U.S. Application Data
(63) Continuation-in-part of application No. 17/891,473, filed on Aug. 19, 2022.

(51) Int. Cl. A61B 6/00

(52) U.S. Cl. CPC A61B 6/482; A61B 6/40; A61B 6/52

(57) ABSTRACT

An energy-dispersive x-ray spectroscopy (EDX) sensing unit, the EDX sensing unit include a protective unit and an x-ray sensor that includes one or more sensing regions. The protective unit is configured to (i) introduce a change in one or more properties of electrons emitted from a sample, thereby preventing the electrons emitted from the sample from reaching the one or more sensing regions, the electrons are emitted from the sample due to an illuminating of the sample by a primary electron beam, and (ii) increase a safety of operation of the EDX sensing unit. The x-ray sensor is configured to (i) receive, by the one or more sensing regions, x-ray photons emitted from the sample due to the illuminating of the sample, and (ii) generate detection signals indicative of the x-ray photons.

### CROSS-REFERENCES TO RELATED APPLICATIONS

This application is a continuation-in-part of U.S. patent application Ser. No. 17/891,473, filed Aug. 19, 2022, the contents of which are hereby incorporated by reference in its entirety.

### BACKGROUND OF THE INVENTION

Energy dispersive x-ray spectroscopy (EDX) is an analytic technique used for the chemical characterization or for the elemental analysis of a sample.

A high output count rate EDX requires to maximize the collection solid angle of the x-ray detector.

A sample is illuminated with a primary electron beam. The illumination causes the sample to emit x-ray photons but also to emit electrons. The electrons may alter the reading and possibly damage the x-ray sensor.

There is a growing need to provide an EDX sensing unit that may protects the x-ray sensor from impinging electrons while maintaining a high solid angle, causing minimal distortion to the photon energetic spectrum, and preventing hazards that are associated with high voltages, for example, voltages that may exceed 500, 1000, 1500, 2000, 4000 volts and more.

### BRIEF SUMMARY OF THE INVENTION

There may be provided an energy-dispersive x-ray spectroscopy (EDX) sensing unit, the EDX sensing unit includes a protective unit and an x-ray sensor that includes one or more sensing regions. The protective unit is configured to (i) introduce a change in one or more properties of electrons emitted from a sample, thereby preventing the electrons emitted from the sample from reaching the one or more sensing regions, the electrons are emitted from the sample due to an illuminating of the sample by a primary electron beam, and (ii) increase a safety of operation of the EDX sensing unit. The x-ray sensor is configured to (i) receive, by the one or more sensing regions, x-ray photons emitted from the sample due to the illuminating of the sample, and (ii) generate detection signals indicative of the x-ray photons.

There may be provided a method for energy-dispersive x-ray spectroscopy (EDX) sensing, the method includes (a) increasing a safety of operation of the EDX sensing unit; (b) illuminating a sample with a primary electron beam, wherein the illuminating causes the sample to emit electrons and x-ray photons; (c) introducing, by a protective unit, a change in one or more properties of the electrons, thereby preventing the electrons from reaching one or more sensing regions of a x-ray sensor; (d) receiving by the one or more sensing regions, the x-ray photons; and (e) generating, by the x-ray sensor, detection signals indicative of the x-ray photons received by the one or more sensing regions.

### BRIEF DESCRIPTION OF THE DRAWINGS

The subject matter regarded as the embodiments of the disclosure is particularly pointed out and distinctly claimed in the concluding portion of the specification. The embodiments of the disclosure, however, both as to organization and method of operation, together with objects, features, and advantages thereof, may best be understood by reference to the following detailed description when read with the accompanying drawings in which:

FIG. 1 is an example of an EDX sensing unit;

FIG. 2 is an example of one or more parts of a EDX sensing unit;

FIG. 3 is an example of one or more parts of a EDX sensing unit;

FIG. 4 is an example of one or more parts of a EDX sensing unit;

FIG. 5 is an example of one or more parts of a EDX sensing unit; and

FIG. 6 is an example of a method.

### DETAILED DESCRIPTION OF THE INVENTION

In the following detailed description, numerous specific details are set forth in order to provide a thorough understanding of the embodiments of the disclosure.

However, it will be understood by those skilled in the art that the present embodiments of the disclosure may be practiced without these specific details. In other instances, well-known methods, procedures, and components have not been described in detail so as not to obscure the present embodiments of the disclosure.

The subject matter regarded as the embodiments of the disclosure is particularly pointed out and distinctly claimed in the concluding portion of the specification. The embodiments of the disclosure, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the disclosure may for the most part, be implemented using electronic components and circuits known to those skilled in the art, details will not be explained in any greater extent than that considered necessary as illustrated above, for the understanding and appreciation of the underlying concepts of the present embodiments of the disclosure and in order not to obfuscate or distract from the teachings of the present embodiments of the disclosure.

The term “and/or” means additionally or alternatively.

There is provided an energy-dispersive x-ray spectroscopy (EDX) sensing unit that includes an x-ray sensor that is protected from electrons, has a large solid angle, and does not substantially distort the energy spectrum of photons reaching the x-ray sensor.

The large solid angle requires the x-ray sensor to be close to the sample and around the primary beam and thus may require preventing energetic electrons leaving the wafer or the sample under test from hitting the x-ray sensor.

The x-ray sensor has one or more sensing regions. The EDX sensing unit may include a protective unit that includes a voltage biased electrode that is configured to introduce a change in one or more properties of electrons emitted from the sample, thereby preventing the electrons emitted from the sample from reaching the one or more sensing regions.

The one or more properties may be speed or trajectory. The voltage biased electrode also has an opening through which electrons may pass and reach one or more electron detectors.

The voltage biased electrode is small (for example—may have a height and/or width of millimetric scale) and is also biased with a high voltage bias voltage. The voltage biased electrode may be proximate (located within a few millimeters) to grounded elements—or elements that are set to significantly different voltage levels.

There is provided a protective unit that improves the safety of the EDX sensing unit—thereby reducing the risk of arcing due to the small distances between the voltage biased electrode and grounded elements (or elements that are set to significantly different voltage levels than the voltage biased electrode)—and also eliminates disturbances of the primary beam due to unwanted accumulation of charges

The protective unit has a voltage biased electrode that has an interior surface and an exterior surface. The interior surface define the aperture—or at least faces the aperture. The exterior surface may be opposite to the interior surface—or at least faces away from the aperture.

The protective unit may include or may exhibit at least one of the following: Has a charge dissipative dielectric that may coat at least one part of at least one element of the protective unit.
Has an x-ray sensor shielding element.
Has an arcing reduction element between a proximal end of the x-ray sensor shielding element and a proximal end of the voltage biased electrode. The arcing reduction element may be a grounded wire or the charge dissipative dielectric.

The suggested EDX sensing unit improves photons detection—for example improves the x-ray photon detection over an entire energy range (up to a primary electron beam energy), with a significant benefit at energies below 1 keV.

There may be provided an EDX sensing unit that may include (a) an x-ray sensor comprising one or more sensing regions; and (b) a protective unit that is configured to (i) introduce a change in one or more properties of electrons emitted from a sample, thereby preventing the electrons emitted from the sample from reaching the one or more sensing regions; wherein the electrons are emitted from the sample due to an illuminating of the sample by a primary electron beam, and (ii) increase a safety of operation of the EDX sensing unit. The x-ray sensor is configured to (i) receive, by the one or more sensing regions, x-ray photons emitted from the sample due to the illuminating of the sample, and (ii) generate detection signals indicative of the x-ray photons.

The one or more properties may include speed and trajectory.

The protective unit may include a voltage biased electrode.

The protective unit may include a x-ray sensor shielding element.

The X-ray sensor shielding element may be a grounded electrode.

The protective unit may include a dielectric material that coats at least a portion of the x-ray sensor shielding element. The dielectric material may be a charge dissipative dielectric material, or an isolating dielectric material.

The voltage biased electrode may include an interior surface, an exterior surface, and a proximal end.

The dielectric material may be a charge dissipative dielectric material that coats the interior surface, the exterior surface, and the proximal end of the voltage biased electrode.

The dielectric material may be a charge dissipative dielectric material that coats a part of the exterior surface, does not coat the interior surface, and does not coat the proximal end.

The dielectric material may be a charge dissipative dielectric material. A part of the charge dissipative dielectric material may be positioned between a part of the exterior surface and the x-ray sensor shielding element.

The EDX sensing unit may include a wire that includes a grounded conductive core that is surrounded by a dielectric shield, wherein the wire contacts a proximal end of the x-ray sensor shielding element.

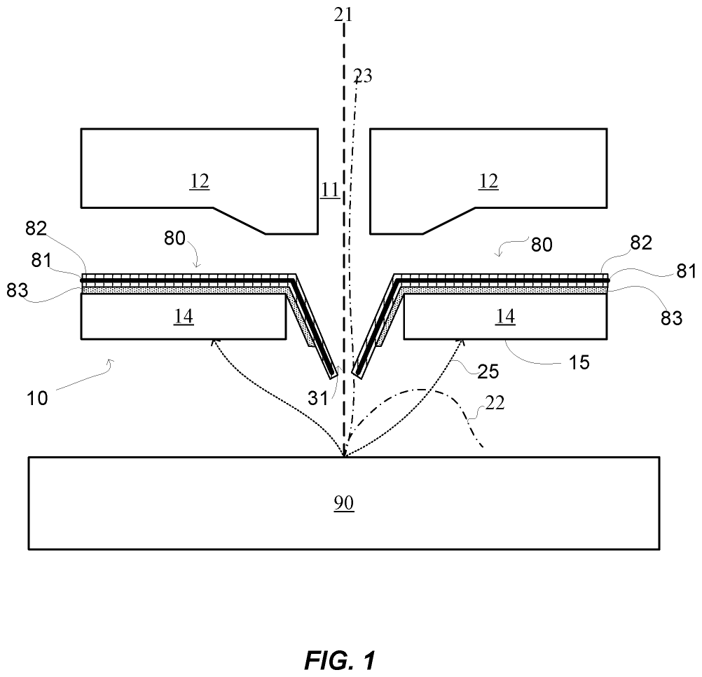

FIG. 1 illustrate an example of a sample 90, EDX sensing unit 10 and an objective lens 12 of a column of an EDX system such as a scanning electron microscope configured to perform EDX measurements. FIG. 1 and other figures may be in scale or may be out of scale.

The EDX sensing unit 10 includes x-ray sensor 14 and protective unit 80.

The x-ray sensor 14 includes sensing region 15 which is the lower surface of the x-ray sensor 14.

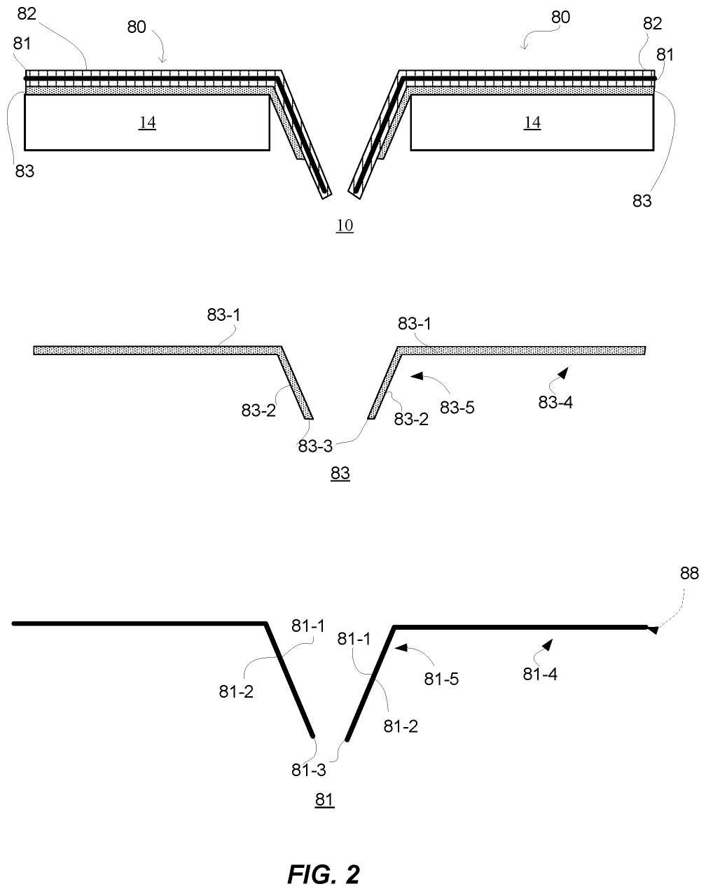

Protective unit 80 includes a voltage biased electrode (VBE) 81 that is biased by bias voltage (denoted 88 in FIG. 2) and introduces an electrostatic field. A voltage difference between the sample 90 and the x-rays sensor 14 prevents electrons 22 from reaching the x-ray sensor. Electrons 23 propagate through opening 31 defined by the VBE 81, and an objective lens opening 11 that passes through objective lens 12.

The protective unit 80 also includes x-ray sensor shielding element (XSSE) 83 and charge dissipative dielectric 82 that coats a VBE interior surface, a VBE exterior surface and a VBE proximal end. The charge dissipative dielectric 82 that coats the VBE exterior surface is located between the VBE exterior surface and the XSSE.

FIG. 2 illustrates the EDS sensing unit 10 of FIG. 1.

The charge dissipative dielectric 82 coats the voltage biased electrode 81 from both sides and also coats the proximal end of the voltage biased electrode 81. The proximal end of the XSSE 83 is higher than the proximal end of the voltage biased electrode 81.

Using a charge dissipative dielectric 82 to coat the voltage biased electrode 81 reduces the chances of a formation of unwanted charges that may distort the primary beam.

FIG. 2 also illustrates the VBE as including VBE interior surface 81-1, VBE exterior surface 81-2, VBE proximal end 81-3, VBE proximal portion 81-5 and VBE distal portion 81-4.

The VBE proximal portion 81-5 is oriented to the VBE distal portion 81-4.

FIG. 2 also illustrates the XSSE as including XSSE interior surface 83-1, XSSE exterior surface 83-2, XSSE proximal end 83-3, XSSE proximal portion 83-5 and XSSE distal portion 83-4. The XSSE proximal portion 83-5 is oriented to the XSSE distal portion 83-4.

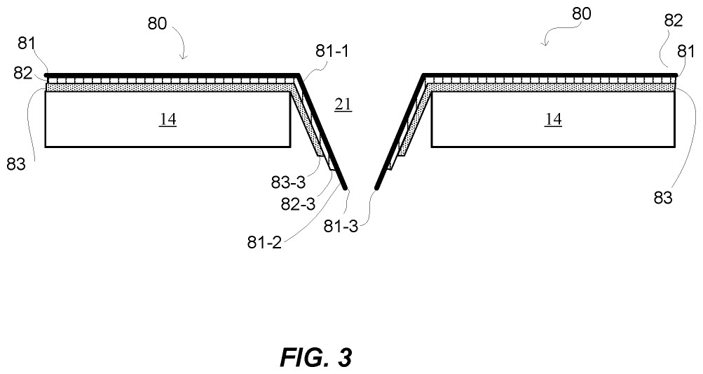

FIG. 3 illustrates an example of an EDS sensing unit 10.

The protective unit includes charge dissipative dielectric 82, VBE 81 and XSSE 83.

The charge dissipative dielectric 82 coats a part of the exterior surface of the voltage biased electrode while leaving the proximal end of the voltage biased electrode, the interior surface of the voltage biased electrode and an uncoated part of the exterior surface of the voltage biased electrode exposed.

The proximal end of the XSSE 83 is higher than a proximal end of the uncoated part.

The proximal end of the uncoated part is higher than the proximal end of the voltage biased electrode 81.

Exposing the interior surface of the voltage biased electrode 81 further reduces the chances of a formation of unwanted charges that may distort the primary beam. The exposer also increases the sensitivity of the EDX sensing unit.

FIG. 3 illustrates an example of an EDX sensing unit 10.

The protective unit includes charge dissipative dielectric 82, VBE 81, XSSE 83 and a grounded wire 85 that includes a grounded conductive core 86 that is surrounded by a dielectric shield 87.

The charge dissipative dielectric 82 coats a part of the exterior surface of the voltage biased electrode while leaving the proximal end of the voltage biased electrode, the interior surface of the voltage biased electrode and an uncoated part of the exterior surface of the voltage biased electrode exposed.

The proximal end of the XSSE 83 is higher than a proximal end of the uncoated part.

The proximal end of the uncoated part is higher than the proximal end of the voltage biased electrode 81.

The grounded wire 85 is located between the proximal end of the XSSE 83 and the proximal end of the uncoated part.

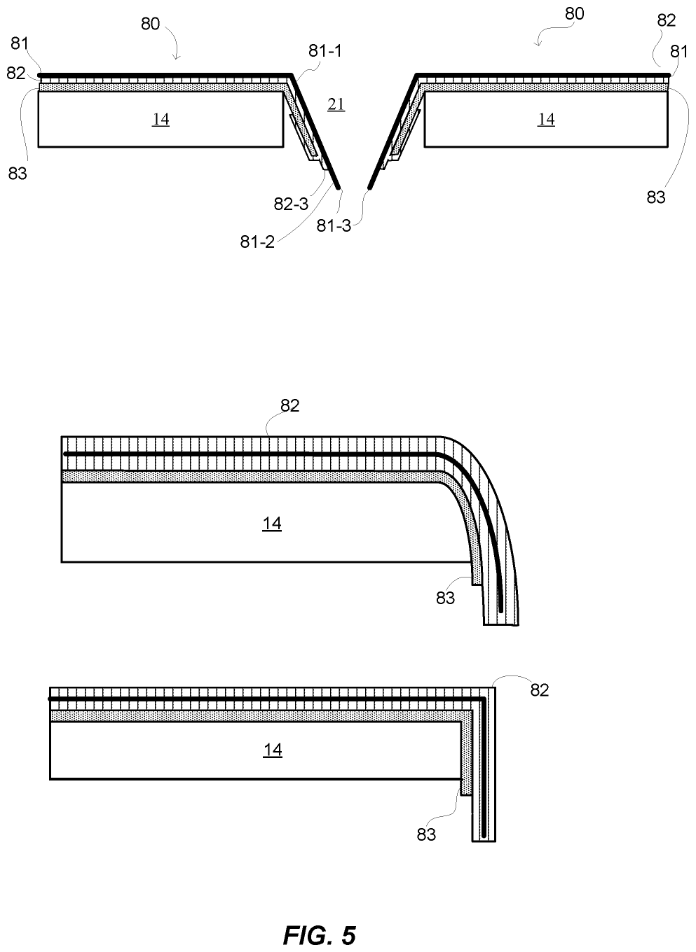

FIG. 5 illustrates and example of an EDX sensing unit 10 and also include examples of parts of the EDX sensing unit

The protective unit includes charge dissipative dielectric 82, VBE 81 and XSSE 83.

The charge dissipative dielectric 82 coats a part of the exterior surface of the voltage biased electrode while leaving the proximal end of the voltage biased electrode, the interior surface of the voltage biased electrode and an uncoated part of the exterior surface of the voltage biased electrode exposed. The charge dissipative dielectric 82 also coats a part of the XXSE exterior surface and coats a proximal end of the XXSE.

The voltage biased electrode and other parts of the protective unit may be of any shape. FIG. 5 also illustrate voltage biased electrode 81 of other shapes—such as a curved voltage biased electrode 81 and voltage biased electrode 81 that has a vertical proximal portion. It should also be noted that the x-ray sensor may be of any shape. There may be any spatial relationship between the x-ray sensor and the protective unit.

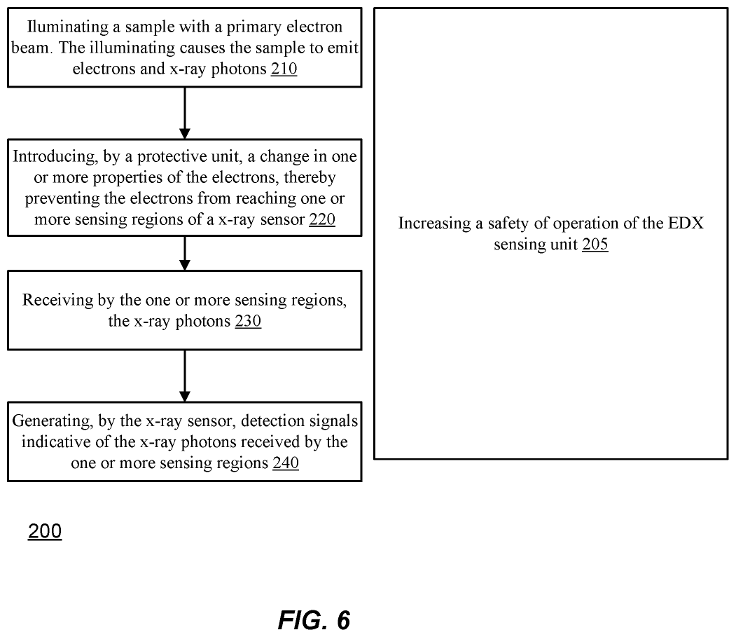

FIG. 6 is an example of method 200 for EDX sensing.

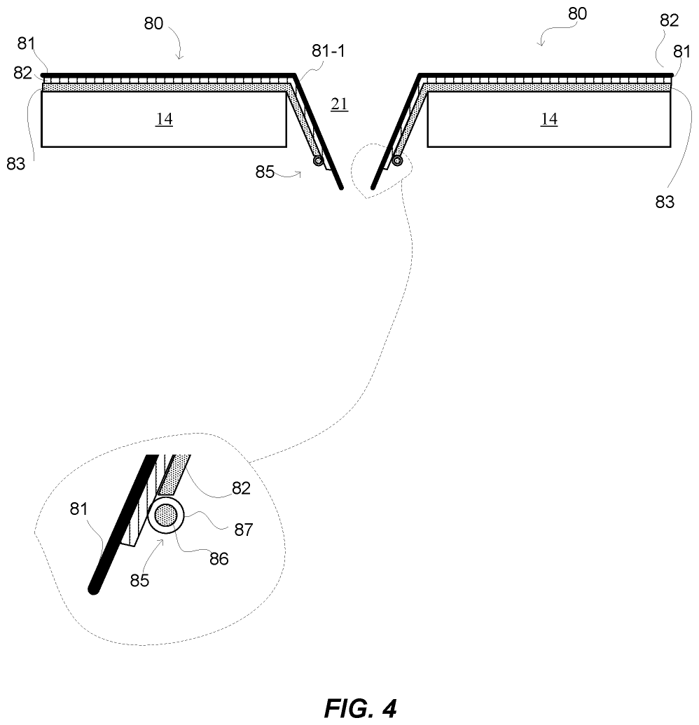

Method 200 may be executed by any of the EDX sensing units illustrated in any one of FIGS. 1, 2, 3, 4 and 5.

Method 200 may include step 205 of increasing a safety of operation of the EDX sensing unit. Step 205 may include reducing the chances of arcing—for example by using any of the protective units and/or any parts of the protective unit illustrated in FIGS. 1-4.

Method 200 may also include step 210 of illuminating a sample with a primary electron beam. The illuminating causes the sample to emit electrons and x-ray photons.

Step 210 may be followed by step 220 of introducing, by the protective unit, a change in one or more properties of the electrons, thereby preventing the electrons from reaching one or more sensing regions of a x-ray sensor. The x-ray sensor may have a solid angle that exceeds 1 steradian. Yet for another example—the x-ray sensor may have a solid angle that does not exceeds 1 steradian.

Step 220 may be followed by step 230 of receiving by the one or more sensing regions, the x-ray photons.

Step 220 may be followed by step 240 of generating, by the x-ray sensor, detection signals indicative of the x-ray photons received by the one or more sensing regions.

The one or more properties may include speed and/or trajectory.

Method 200 may be executed using any of the EDX sensing units and/or any of the protective units illustrated in FIGS. 1-4.

For example:
The protective unit may include a voltage biased electrode.
The protective unit may include a x-ray sensor shielding element.
The X-ray sensor shielding element may be a grounded electrode.
The protective unit may include a dielectric material that coats at least a portion of the x-ray sensor shielding element. The dielectric material may be a charge dissipative dielectric material, or an isolating dielectric material.
The voltage biased electrode may include an interior surface, an exterior surface, and a proximal end.
The dielectric material may be a charge dissipative dielectric material that coats the interior surface, the exterior surface, and the proximal end of the voltage biased electrode.
The dielectric material may be a charge dissipative dielectric material that coats a part of the exterior surface, does not coat the interior surface, and does not coat the proximal end.
The dielectric material may be a charge dissipative dielectric material. A part of the charge dissipative dielectric material may be positioned between a part of the exterior surface and the x-ray sensor shielding element.
The EDX sensing unit may include a wire that includes a grounded core that is surrounded by a dielectric shield, wherein the wire contacts a proximal end of the x-ray sensor shielding element.

In the foregoing specification, the embodiments of the disclosure has been described with reference to specific examples of embodiments of the disclosure. It will, however, be evident that various modifications and changes may be made therein without departing from the broader spirit and scope of the embodiments of the disclosure as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the disclosure described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to be a single connection, a plurality of connections, unidirectional connections, or bidirectional connections. However, different embodiments may vary the implementation of the connections.

For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above-described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to embodiments of the disclosures containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the embodiments of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the embodiments of the disclosure.

What is claimed is:

1. An energy-dispersive x-ray spectroscopy (EDX) sensing unit, comprising:
an x-ray sensor comprising one or more sensing regions; and
a protective unit that is configured to (i) introduce a change in one or more properties of electrons emitted from a sample, thereby preventing the electrons emitted from the sample from reaching the one or more sensing regions; wherein the electrons are emitted from the sample due to an illuminating of the sample by a primary electron beam, and (ii) increase a safety of operation of the EDX sensing unit;
wherein the x-ray sensor is configured to (i) receive, by the one or more sensing regions, x-ray photons emitted from the sample due to the illuminating of the sample, and (ii) generate detection signals indicative of the x-ray photons.

2. The EDX sensing unit according to claim 1 wherein the one or more properties comprise speed and trajectory.

3. The EDX sensing unit according to claim 1, wherein the protective unit comprises a voltage biased electrode.

4. The EDX sensing unit according to claim 3, wherein the protective unit comprises an x-ray sensor shielding element.

5. The EDX sensing unit according to claim 4, wherein the x-ray sensor shielding element is a grounded electrode.

6. The EDX sensing unit according to claim 4, comprising a dielectric material that coats at least a portion of the x-ray sensor shielding element.

7. The EDX sensing unit according to claim 6 wherein the dielectric material is a charge dissipative dielectric material.

8. The EDX sensing unit according to claim 6, wherein the voltage biased electrode comprises an interior surface, an exterior surface, and a proximal end.

9. The EDX sensing unit according to claim 8, wherein the dielectric material is a charge dissipative dielectric material that coats the interior surface, the exterior surface, and the proximal end.

10. The EDX sensing unit according to claim 8, wherein the dielectric material is a charge dissipative dielectric material that coats a part of the exterior surface, does not coat the interior surface, and does not coat the proximal end.

11. The EDX sensing unit according to claim 8, wherein the dielectric material is a charge dissipative dielectric material, a part of the charge dissipative dielectric material is positioned between a part of the exterior surface and the x-ray sensor shielding element.

12. The EDX sensing unit according to claim 8, comprising a wire that comprises a grounded conductive core that is surrounded by a dielectric shield, wherein the wire contacts a proximal end of the x-ray sensor shielding element.

13. The EDX sensing unit according to claim 8, comprising a grounded conductive core that is surrounded by a dielectric shield.

14. A method for energy-dispersive x-ray spectroscopy (EDX) sensing, the method comprises:
increasing a safety of operation of an EDX sensing unit;
illuminating a sample with a primary electron beam, wherein the illuminating causes the sample to emit electrons and x-ray photons;
introducing, by a protective unit, a change in one or more properties of the electrons, thereby preventing the electrons from reaching one or more sensing regions of a x-ray sensor;
receiving by the one or more sensing regions of an x-ray sensor of the EDX sensing unit, the x-ray photons; and
generating, by the x-ray sensor, detection signals indicative of the x-ray photons received by the one or more sensing regions.

15. The method according to claim 14 wherein the one or more properties comprise speed and trajectory.

16. The method according to claim 14, wherein the protective unit comprises a voltage biased electrode.

17. The method according to claim 16, wherein the protective unit comprises an x-ray sensor shielding element.

18. The method according to claim 17, wherein the x-ray sensor shielding element is a grounded electrode.

19. The method according to claim 17, comprising a dielectric material that coats at least a portion of the x-ray sensor shielding element.

20. The method according to claim 19 wherein the dielectric material is a charge dissipative dielectric material.
